(19) United States
(12) Patent Application Publication
(10) Pub. No.: US 2014/0350354 A1
(43) Pub. Date: Nov. 27, 2014

(54) Oral Appliance Monitor and Method of Using the Same

(71) Applicant: 12th Man Technologies, Inc., Garden Grove, CA (US)

(72) Inventors: Alex Stenzler, Long Beach, CA (US); William Ross, Las Vegas, NV (US); Steve Han, Huntington Beach, CA (US); Jeffory Wyscarver, Corona, CA (US)

(73) Assignee: 12th Man Technologies, Inc., Garden Grove, CA (US)

(21) Appl. No.: 13/960,239

(22) Filed: Aug. 6, 2013

Related U.S. Application Data
(60) Provisional application No. 61/827,526, filed on May 24, 2013.

(51) Int. Cl. A61B 5/00; A61F 5/56; A61B 7/00; A61B 5/113; A61B 5/087; A61B 5/01

(52) U.S. Cl. CPC A61B 5/4818; A61B 5/087; A61B 5/01; A61B 7/003; A61B 5/113; A61B 5/6814; A61F 5/566; A61F 2005/563
USPC 600/301; 600/538; 600/549; 600/586; 600/595; 128/848

(57) ABSTRACT

An oral appliance assembly is described. The assembly includes an oral appliance component having an upper teeth tray and a lower teeth tray. The assembly further includes a module releasably connected to the oral appliance. The module includes at least one sensor that is positioned outside of the mouth and underneath the nares of a subject's nose when the oral appliance component is positioned in the subject's mouth. A method of measuring user compliance of an oral appliance is also described. The method includes positioning an oral appliance in the mouth of a subject, measuring at least one parameter of airflow from the subject's nose or mouth, and determining compliance based on the at least one measured parameter. A method of measuring effectiveness of an oral appliance is also described. The method includes positioning an oral appliance in the mouth of a subject, measuring at least one parameter of airflow from the subject's nose or mouth, and determining effectiveness based on the at least one measured parameter.

### CROSS-REFERENCE TO RELATED APPLICATIONS

The present application claims priority under 35 U.S.C. 119(e) to U.S. Provisional Patent Application No. 61/827,526, filed May 24, 2013, which is herein incorporated by reference in its entirety.

### BACKGROUND OF THE INVENTION

Various kinds of oral appliances are frequently prescribed for the treatment of sleep apnea, for nocturnal bruxism (grinding or gnashing of teeth) or for relief of snoring. For many patients this type of therapy is effective, as the adjustment made when wearing an oral appliance titrates the forward movement of the lower jaw (mandible) to open the airway, addressing the conditions of snoring or sleep apnea. In the case of bruxism, vertical distance is created, thereby preventing occlusions from occurring or reducing the strength of force exerted on the teeth. However, correct positioning is key to the effectiveness of the oral appliance. For example, in the case of a sleep apnea appliance, if there is too little forward movement, the obstruction remains, and if there is too much movement, the oral appliance can cause pain and discomfort, occasionally resulting in malocclusion or mandibular joint dysfunction.

In many cases, to determine the correct positioning of the sleep apnea oral appliance, multiple sleep studies are required so as to know which position provides the optimal airway patency. Unfortunately, these studies take a considerable amount of time and are typically expensive. An additional limitation of these “one-time” studies is that for most people, the degree of obstruction may change with changes in body weight, degree of sleep deprivation and amount of alcohol consumed. A well-documented variable with regards to disordered breathing and bruxism is night to night variability, which is managed by monitoring over time and documenting breathing trends over several nights. Therefore it would be beneficial for the existence of a device and method that could record the effectiveness of oral appliances on a routine or continuous basis. For snoring or bruxism, oral appliances are frequently prescribed, but without any measurement of the effectiveness of the oral appliance. Further, there is the potential risk that use of an oral appliance applied for bruxism or snoring, may actually increase the risk of obstructive sleep apnea.

Another problem with existing oral appliances relates to determining the compliance of a patient in wearing the oral appliance as prescribed. The most frequent treatment for sleep apnea is nasal continuous positive airway pressure (CPAP). While it is very effective, compliance with using CPAP is typically below 50% within just six months of prescription. Measurements of adherence to use of CPAP and all other forms of airway treatment are important for both medical and insurance purposes. While oral appliance adherence appears to be better than with CPAP, accuracy of such measures is lacking and relies primarily on anecdotal evidence, as methods for determining such adherence are significantly limited. With CPAP, breath-by-breath data is stored and can be reviewed by a medical expert to assure appropriate treatment. Without solid evidence of proper positioning and compliance, acceptance of oral appliances as an effective therapy is still in question by the medical community.

One application for an oral appliance monitoring device to collect data on the compliance of their use is described by Moore (PCT WO 2012/064684 A2). By using pressure transducers in the trays for the teeth, Moore describes measurement of oral forces to determine whether the oral appliance is in the patient's mouth. This method, while recording compliance, does not provide any indication of the effectiveness of positioning.

Thus, there is a need in the art for an oral appliance that monitors both the effectiveness of the positioning of the oral appliance and compliance of use. The present invention satisfies this need.

### SUMMARY OF THE INVENTION

The present invention relates to devices, systems, and methods related to oral appliances and measuring the effectiveness and/or user compliance of oral appliances. In one embodiment, the present invention relates to an oral appliance assembly, comprising: an oral appliance component including an upper teeth tray and a lower teeth tray; a module releasably connected to the oral appliance including at least one sensor; wherein the at least one sensor is positioned outside of the mouth and underneath the nares of a subject's nose when the oral appliance component is positioned in the subject's mouth. In another embodiment, the present invention relates to an oral appliance, comprising: an upper teeth tray and a lower teeth tray; a module including at least one sensor; wherein at least one sensor of the module is positioned outside of the mouth and underneath the nares of a subject's nose when the upper and lower teeth trays are positioned in the subject's mouth.

In various embodiments, the at least one sensor of the oral appliance, oral appliance component, or oral appliance assembly is selected from the group consisting of an accelerometer, a pressure transducer, an acoustic sensor, a thermistor and a thin-film resistive flow sensor. In one embodiment, the oral appliance assembly, or the at least one sensor of the oral appliance component, measures at least one parameter of airflow from the subject's nose or mouth selected from the group consisting of temperature, air flow, pressure and movement. In one embodiment, the system of the present invention determines compliance based on the at least one measured parameter. In another embodiment, the system determines compliance based at least one measured parameter from inside the subject's mouth and at least one measured parameter of airflow from the subject's nose.

The module of the oral appliance assembly of the present invention may also further comprise a processor and a memory. In one embodiment, the module is electrically activated only when connected to the oral appliance component. In one embodiment, the module further comprises a wireless transmitter. In one embodiment, the module further comprises a power source. In one embodiment, the module further comprises an induction coil for wireless charging of a battery.

The device of the present invention may further comprise a means for identifying the oral appliance assembly. In one embodiment, the means for identifying the oral appliance assembly is selected from the group consisting of an RFID tag, a microchip, or a resistor with a unique resistance value.

The present invention also relates to a method of measuring user compliance of an oral appliance, comprising: positioning an oral appliance in the mouth of a subject; measuring at least one parameter of airflow, temperature or sound from the subject's nose or mouth; and determining compliance based on the at least one measured parameter. In one embodiment, the at least one parameter is measured by a module mechanically connected to the oral appliance, wherein the module comprises at least one sensor positioned outside the subject's mouth and underneath the subject's nose. In one embodiment, compliance is further determined according to the quality of the subject's breathing. In another embodiment, compliance is further determined according to reaching a threshold value of measured airflow, temperature or sound from the subject's nose or mouth. In one embodiment, the threshold value is based on reaching a target respiratory rate. In another embodiment, the threshold value is based on reaching a target time interval between breaths. In yet another embodiment, the threshold value is based on reaching a target time duration of the oral appliance in the mouth.

The present invention also relates to a method of measuring the effectiveness of an oral appliance, comprising: positioning an oral appliance in the mouth of a subject; measuring at least one parameter of airflow, temperature or sound from the subject's nose or mouth; and determining effectiveness based on the at least one measured parameter. In one embodiment, the at least one parameter is measured by a module mechanically connected to the oral appliance, wherein the module comprises at least one sensor positioned outside the subject's mouth and underneath the subject's nose. In one embodiment, effectiveness is further determined according to reaching a threshold value of measured airflow, temperature or sound from the subject's nose or mouth. In one embodiment, the threshold value is based on reaching a target respiratory rate. In another embodiment, the threshold value is based on reaching a target time interval between breaths.

In various embodiments, the systems and methods of the present invention include the ability to enter a low power consumption mode. In one embodiment, the module, the oral appliance, or both, enters a low power consumption mode when no value for measured airflow, temperature, movement, or sound is detected from the subject's nose or mouth within a specified detection time period. In one embodiment, the specified detection time period is one hundred and twenty (120) seconds. In another embodiment, the low power consumption mode is maintained for a specified snooze time period. In one embodiment, the specified snooze time period is fifteen (15) minutes. In another embodiment, the low power consumption mode is maintained until motion by the subject is detected by an accelerometer. In yet another embodiment, the low power consumption mode is maintained until breathing by the subject is detected by the sensing or detection of air flow.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A and 1B, is a schematic of exemplary embodiments of an oral appliance assembly of the present invention.

FIG. 2, comprising FIGS. 2A and 2B, is a schematic of the embodiments of FIG. 1 positioned in a subject's mouth.

FIG. 3 is a front view of the oral appliance assembly with a connection port.

FIG. 4 is a bottom view of an upper teeth tray portion of an oral appliance component with a set of recesses for engaging the arm of an external module.

FIG. 5 is a top view of an exemplary oral appliance assembly.

FIG. 6 is a perspective view of an exemplary oral appliance assembly.

FIG. 7 is chart depicting how a trace of airflow from the subject's nose diminishes during periods where the airway is obstructed, indicating an ineffective positioning of the oral appliance in the subject's mouth.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical oral appliances. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The present invention includes an assembly comprised of an oral appliance component with an external module that is positioned outside the mouth. When the oral appliance component is positioned in the mouth of a subject, the external module is positioned outside the mouth and beneath the nares of the subject's nose, such that the external module can measure one or more attributes of the flow of air from the subject's nostrils or mouth.

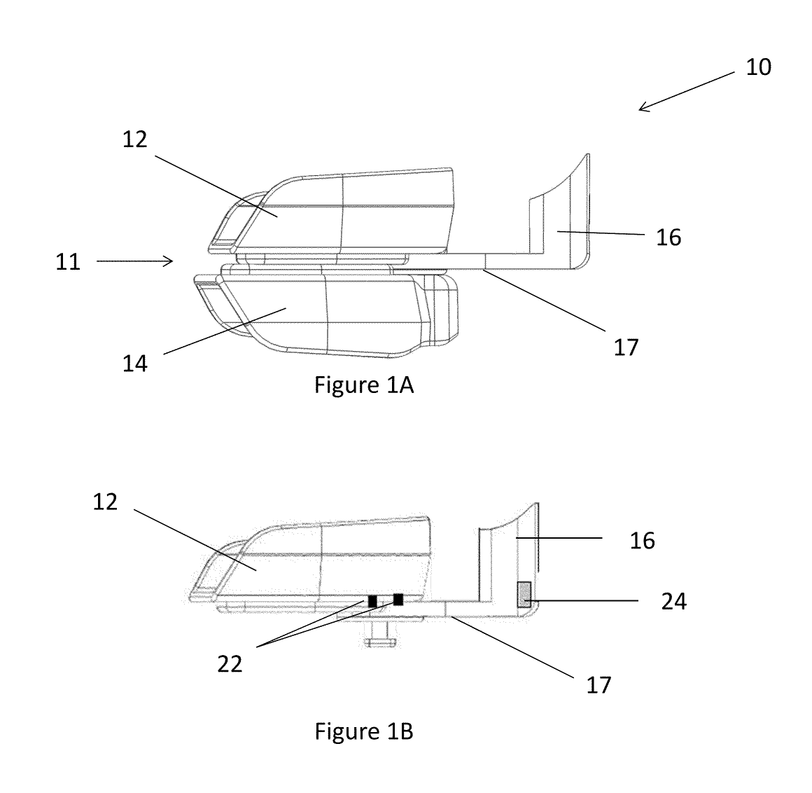

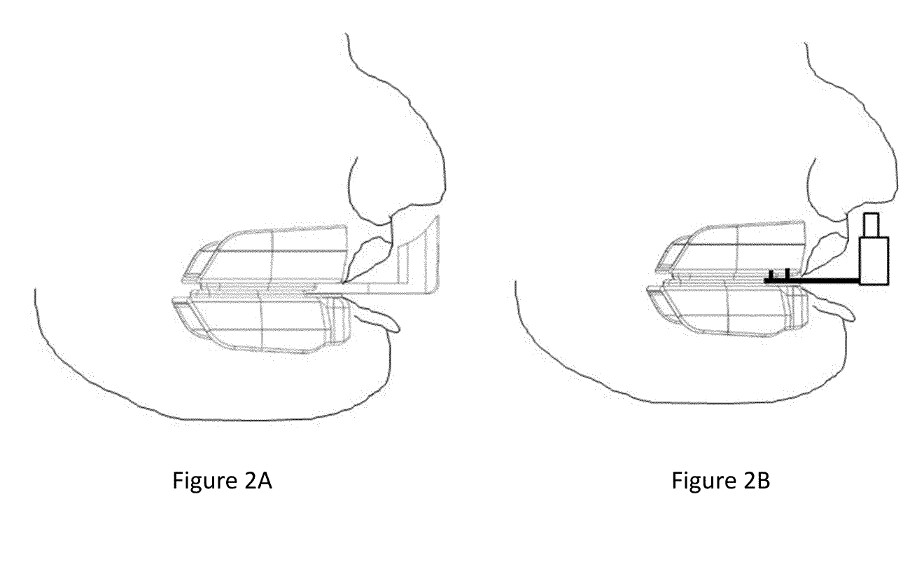

As illustrated in FIGS. 1A and 1B, an assembly 10 generally includes an oral appliance component 11 and an external module 16. Oral appliance component 11 includes an upper teeth tray 12 and a lower teeth tray 14. When oral appliance component 11 is placed in a subject's mouth, as shown in FIGS. 2A and 2B, lower tray 14 may slide or shift relative to upper tray 12 so as to provide an adjustable mandibular advancement. By allowing lower tray 14 to adjust relative to upper tray 12, the subject's lower jaw, tongue, soft palate and hyoid bone may be strategically positioned to create a more open airway, thereby preventing airway closure during sleep. As contemplated herein, upper and lower trays 12 and 14 may include any standard design and construction, such as those found with stand-alone adjustable mandibular advancement devices having upper and lower trays that are well known in the art. Examples of such devices utilizing similar functionality include the Halstrom Hinge, ApneaRX or Narval. Upper and lower trays 12 and 14 may further include any design and/or structural elements promoting additional comfort to the subject's lips or other surrounding soft tissues in contact with upper and lower trays 12 and 14 of oral appliance 10, as would be understood by those skilled in the art.

Oral appliance component 11 may include a connection port for securely connecting an extended arm 17 of external module 16 to oral appliance 11. As contemplated herein, the connection port may allow for the releasable engagement of external module 16 via arm 17, such that external module 16 may be detached as desired by the subject.

In another embodiment as shown in FIG. 4, the attachment mechanism may include recesses 20 in oral appliance component 11 for receiving a set of pins 22 (FIG. 1B) on attachment arm 17 for engaging recesses 20. Recesses 20 may be located on either upper tray 12, lower tray 14, or they may be located on both upper and lower trays 12 and 14. It should be appreciated that the attachment mechanism may be any mechanism understood by those skilled in the art, including detents, friction fittings, couplings, clips, ports and the like. Preferably, external module 16 is releasably attachable to oral appliance component 11, such that external module 16 can be detached when needed. In other embodiments, external module 16 is fixedly attached upon assembly, or otherwise not detachable.

It should be appreciated that the connection of external module 16 via arm 17 to oral appliance component 11 not only provides a secure and releasable attachment, but further may provide an electrical connection, such that oral appliance component 11 is electrically and communicatively connected to external module 16 for the transmission of power and or data as needed. In another embodiment, oral appliance component 11 may provide for the closing of an electrical switch in arm 17 of external module 16, such that external module 16 is not powered or otherwise cannot record or store data unless connected to oral appliance 11. For example, the physical connection can be the closing of a circuit whereby metal tips on arm 17 connect to a metal plate/wire between two of the interlocking recesses, or it can be a microswitch on one of the pins 22 (FIG. 1B) that is pressed closed when inserted into one of the recesses 20 (FIG. 4), or any other technology that can electrically or communicatively link the oral appliance component 11 and external module 16 together.

As previously described herein, external module 16 includes extended arm 17 for engaging upper tray 12 and/or lower tray 14 of oral appliance component 11. Arm 17 may include pins 22 for engaging recesses 20, as shown in FIG. 1B, or it may include any other extension or component suitable for engaging oral appliance component 11.

As shown in FIGS. 1B, 5 and 6, external module 16 also includes a housing with at least one sensor 23 embedded within or extending from the housing. Preferably, external module 16 includes a plurality of sensors for measuring, without limitation, temperature, air flow, pressure, sound or movement. For example, external module 16 may include an accelerometer 24. In one embodiment, accelerometer 24 may be a 3D accelerometer embedded in the housing of external module 16 for monitoring body movement and body position of the subject. In another example, the sensors of external module 16 include pressure transducers, acoustic sensors, thermistors, thin-film resistive flow sensors and any combination thereof. Exemplary sensors include those as manufactured from Sensirion, Silicone Microstructures, Audio-Technica, DuPont and Kapton. Measurements from these sensors may be used to calculate parameters such as respiratory rate, intervals between breaths, and the duration of the oral appliance in the mouth. The acoustic sensors (microphones, pressure transducers, accelerometers and thin film sensors) may be used to record snoring or grinding of the teeth or respiration.

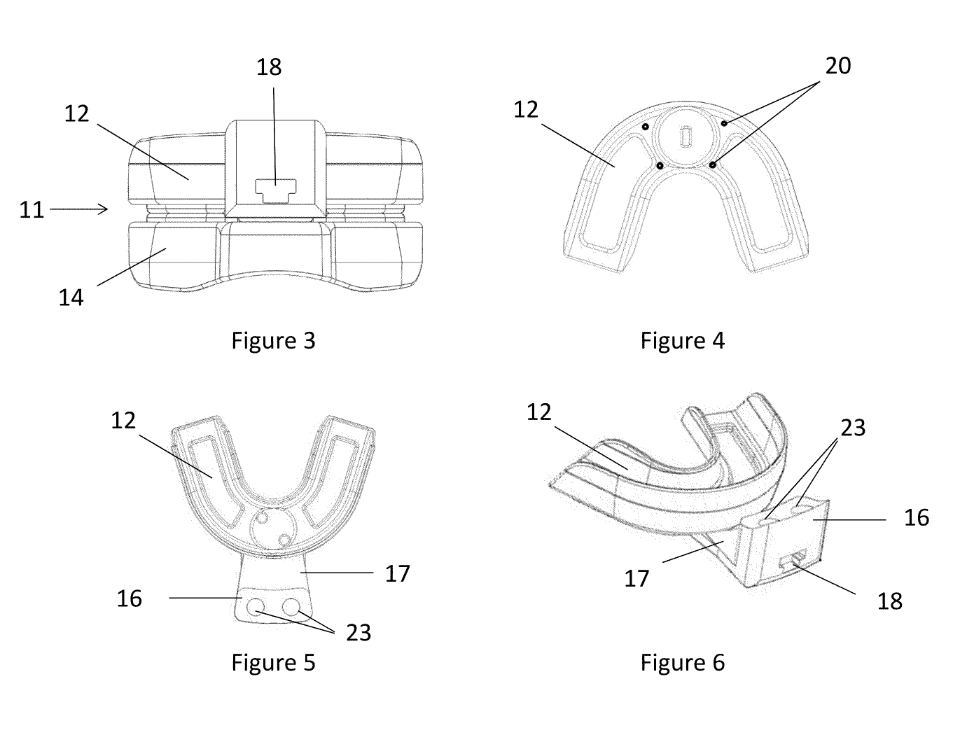

External module 16 may further include a power source, such as a battery, and may further include a volatile and/or non-volatile local memory, a wireless transmitter or transceiver, a processor, a signal converter, an oscillator and any circuitry needed to power the device, to collect and store data from the sensors, and to transmit such data to a secondary computing device. External module 16 may also include any sort of data port 18 (FIGS. 3 and 6) or connector for charging the battery of external module 16, powering external module 16 via an external power source, or for transferring data to/from a secondary computing device. In one embodiment, external module 16 includes a means for charging the battery by induction, i.e., wireless charging, thereby eliminating the need for a physical connector in order to charge the battery. In such an embodiment, the external module may include an induction coil for converting power from an electromagnetic field into electrical current to charge the battery.

In certain embodiments, oral appliance component 11 may also include one or more sensors for measuring, without limitation, temperature, air flow, pressure or movement, or contact with a portion of the subject's mouth, such as by the tongue or teeth. Such sensors may include any type of sensor described herein, including pressure transducers, acoustic sensors, thermistors, thin-film resistive flow sensors and any combination thereof. Likewise, oral appliance component 11 may further include a power source, a memory, a transmitter or transceiver, a processor, a signal converter, an oscillator and any circuitry needed to power the component, to collect and store data from the sensors, and to transmit such data to the external module or to a secondary computing device.

In embodiments in which external module 16 is detachable from oral appliance component 11, external module 16 may be interchanged among multiple subjects. In such embodiments, oral appliance component 11 may include a means of identifying the oral appliance to distinguish one subject's oral appliance from another subject's oral appliance. For example, oral appliance 11 may include an embedded radio-frequency identification (RFID) tag, such as a Hitachi “Powder” RFID microchip, while external module 16 includes a RFID reader positioned to read the embedded RFID tag in oral appliance 11. In other examples, oral appliance 11 may simply include any type of microchip known in the art for storing information specific to oral appliance 11 and/or to a particular subject. In another example, oral appliance 11 may include a resistor with a unique value that can be sensed by external module 16 when external module 16 is electrically connected to oral appliance component 11. It should be appreciated that the present invention is not limited to any particular mechanism for communication and identification between the oral appliance and external module.

It should be appreciated that, unlike existing oral appliances, the present invention uniquely integrates an oral, mandibular adjustment component with an external module for measurement of exhaled air from the subject's nose or mouth. By having both components together, it is possible to determine not only if the subject is in compliance with wearing the oral appliance component, but also if the subject is wearing the oral appliance component effectively, such that the subject's airways are properly opened as measured by airflow from the nose or mouth. For example, when the external module is attached to the oral appliance component, the external module is activated, and senses respiration via the flow of air or sound exiting the nostrils or mouth. In this manner, the module collects data pertaining to respiratory frequency, periods of snoring, apnea and reduced ventilation, body position, body movement and the like, and stores this data in a local memory. When the subject is done wearing the oral appliance component of the assembly, the external module can be attached to a reader to download the collected data. In another embodiment, the module can be disconnected from the oral appliance component for connection to a reader, or alternatively, the data can be transferred wirelessly to a secondary computing device, such as via Bluetooth.

In another embodiment, the present invention is a single unit, integrating both the oral appliance component and external module as one piece. In other words, the external module portion is not detachable from the oral appliance portion. In such embodiments, the single unit may include a power source, local memory, wireless transmitter or transceiver, processor, signal converter, oscillator, sensors and any circuitry needed to power the device, to collect and store data from the sensors, and to transmit such data to a secondary computing device. Such embodiments will also include any sort of data port or connector for charging the battery of the single unit, powering the single unit via an external power source, or for transferring data to/from a secondary computing device.

The present invention includes a method of measuring user compliance of an oral appliance. The method includes positioning an oral appliance in the mouth of a subject, sensing at least one parameter of airflow from the subject's nose or mouth, or movement and determining compliance based on the at least one sensed parameter. In other embodiments, the method includes sensing at least one parameter of airflow from the subject's nose or mouth, as well as at least one parameter from inside the subject's mouth. As contemplated herein, compliance may be additionally based or scored according to the quality of the subject's breathing. For example, compliance may be determined according to reaching a threshold value of measured airflow from the subject's nose. Exemplary threshold values may be based on reaching a target respiratory rate, the time duration of the oral appliance in the mouth, or any combination of such measured values. Alternatively, compliance may be determined according to reaching a threshold of temperature or breathing sounds.

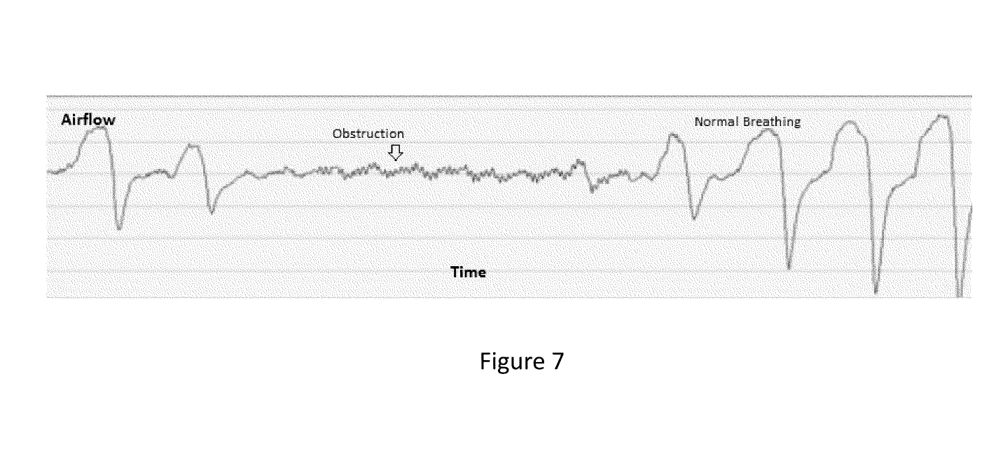

The present invention also includes a method of measuring the effectiveness of an oral appliance. The method includes positioning an oral appliance in the mouth of a subject, sensing at least one parameter of airflow from the subject's nose or mouth, and determining effectiveness based on the at least one sensed parameter. In other embodiments, the method includes sensing at least one parameter of airflow from the subject's nose or mouth, as well as at least one parameter from inside the subject's mouth. As contemplated herein, effectiveness may be additionally based or scored according to the quality of the subject's breathing. For example, effectiveness may be determined according to reaching a threshold value of measured airflow from the subject's nose or mouth. As shown in FIG. 7, a trace of airflow from the subject's nose diminishes during periods where the airway is obstructed indicating that the position of the oral appliance is not correct to maintain airway patency at all times. Exemplary threshold values may be based on reaching a target respiratory rate, a target time interval between breaths, or any combination of such measured values. Alternatively, effectiveness may be determined according to reaching a threshold of temperature or breathing sounds. Alternatively, effectiveness may be determined according to reaching a threshold of sounds or movement from the teeth.

The method of the present invention may also include steps for reducing power consumption wherein the oral appliance component, the external module, or both, enter a low power “sleep” mode during periods of inactivity. In one embodiment, the system of the present invention continuously monitors airflow of a subject as long as activity related to at least one parameter (such as airflow, temperature, sound, or movement, for example) is detected by a sensor, such as an accelerometer, within a specified time duration, for example 120 seconds. If no activity related to an airflow, temperature, sound, or movement parameter is detected for the specified time duration, the system will enter a low power consumption mode. When in a low power or “sleep” mode, the system may return to a normal power consumption mode if a sensor detects activity, for example if movement is detected by an accelerometer. In such a normal power consumption mode, the airflow of the subject will be monitored continuously. In another embodiment, the system will return to a normal power consumption mode after a snooze period has elapsed, for example a period of 15 minutes. In yet another embodiment, the system may return to a normal power mode if either activity is detected, or a snooze period has elapsed. However, the time periods or durations are not limited to those specified herein. In various embodiments, the time duration for detecting activity before the system enters the sleep mode may be some duration other than 120 seconds, for example, a time in the range of about 5 seconds to 5 minutes, such as 30 seconds, 60 seconds, 90 seconds, 150 seconds, or 180 seconds. In one embodiment, the snooze period may be some period other than 15 minutes, for example, a period in the range of about 10 seconds to 2 hours, such as 30 seconds, 1 minute, 2 minutes, 5 minutes, 10 minutes, 20 minutes, 30 minutes, or 1 hour. As contemplated herein, any standard programming for signaling changes in power mode may be used in the present invention, as would be understood by those skilled in the art.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An oral appliance assembly, comprising:
an oral appliance component including an upper teeth tray and a lower teeth tray;
a module releasably connected to the oral appliance including at least one sensor;
wherein the at least one sensor is positioned outside of the mouth and underneath the nares of a subject's nose when the oral appliance component is positioned in the subject's mouth.

2. The oral appliance assembly of claim 1, wherein the at least one sensor is selected from the group consisting of an accelerometer, a pressure transducer, an acoustic sensor, a thermistor and a thin-film resistive flow sensor.

3. The oral appliance assembly of claim 2, wherein the assembly measures at least one parameter of airflow from the subject's nose or mouth selected from the group consisting of temperature, air flow, pressure and movement.

4. The oral appliance assembly of claim 1, wherein the module further comprises a processor and a memory.

5. The oral appliance assembly of claim 4, wherein the module is electrically activated only when connected to the oral appliance component.

6. The oral appliance assembly of claim 5, wherein the assembly determines compliance based on the at least one measured parameter.

7. The oral appliance assembly of claim 4, wherein the module further comprises a wireless transmitter.

8. The oral appliance assembly of claim 4, wherein the module further comprises a power source.

9. The oral appliance assembly of claim 4, wherein the module further comprises an induction coil.

10. The oral appliance assembly of claim 1, wherein the oral appliance component further comprises at least one sensor.

11. The oral appliance assembly of claim 10, wherein the at least one sensor of the oral appliance component is selected from the group consisting of an accelerometer, a pressure transducer, an acoustic sensor, a thermistor and a thin-film resistive flow sensor.

12. The oral appliance assembly of claim 11, wherein the at least one sensor of the oral appliance component measures at least one parameter from inside the subject's mouth selected from the group consisting of temperature, air flow, pressure and movement.

13. The oral appliance assembly of claim 12, wherein the assembly determines compliance based at least one measured parameter from inside the subject's mouth and at least one measured parameter of airflow from the subject's nose.

14. The oral appliance assembly of claim 1, wherein the oral appliance component includes a means for identifying the oral appliance assembly.

15. The oral appliance assembly of claim 14, wherein the means for identifying the oral appliance assembly is selected from the group consisting of an RFID tag, a microchip, and a resistor with a unique resistance value.

16. A method of measuring user compliance of an oral appliance, comprising:
positioning an oral appliance in the mouth of a subject;
measuring at least one parameter of airflow, temperature, movement or sound from the subject's nose or mouth; and
determining compliance based on the at least one measured parameter.

17. The method of claim 16, wherein the at least one parameter is measured by a module mechanically connected to the oral appliance, wherein the module comprises at least one sensor positioned outside the subject's mouth and underneath the subject's nose.

18. The method of claim 17, wherein compliance is further determined according to the quality of the subject's breathing.

19. The method of claim 18, wherein compliance is further determined according to reaching a threshold value of measured airflow, temperature, movement or sound from the subject's nose or mouth.

20. The method of claim 19, wherein the threshold value is based on reaching a target respiratory rate.

21. The method of claim 19, wherein the threshold value is based on reaching a target time interval between breaths.

22. The method of claim 19, wherein the threshold value is based on reaching a target time duration of the oral appliance in the mouth.

23. A method of measuring the effectiveness of an oral appliance, comprising:
positioning an oral appliance in the mouth of a subject;
measuring at least one parameter of airflow, temperature, movement or sound from the subject's nose or mouth; and
determining effectiveness based on the at least one measured parameter.

24. The method of claim 23, wherein the at least one parameter is measured by a module mechanically connected to the oral appliance, wherein the module comprises at least one sensor positioned outside the subject's mouth and underneath the subject's nose.

25. The method of claim 24, wherein effectiveness is further determined according to reaching a threshold value of measured airflow, temperature, movement or sound from the subject's nose or mouth.

26. The method of claim 25, wherein the threshold value is based on reaching a target respiratory rate.

27. The method of claim 25, wherein the threshold value is based on reaching a target time interval between breaths.

28. The method of claim 24, wherein the module, the oral appliance, or both, enters a low power consumption mode when no value for measured airflow, temperature, movement, or sound is detected from the subject's nose or mouth within a specified detection time period.

29. The method of claim 28, wherein the specified detection time period is about one hundred twenty (120) seconds.

30. The method of claim 28, wherein the low power consumption mode is maintained for a specified snooze time period.

31. The method of claim 30, wherein the specified snooze time period is about fifteen (15) minutes.

32. The method of claim 28, wherein the low power consumption mode is maintained until motion by the subject is detected by an accelerometer.

33. The method of claim 28, wherein the low power consumption mode is maintained until breathing by the subject is detected by sensing of air flow.

34. An oral appliance, comprising:
an upper teeth tray and a lower teeth tray;
a module including at least one sensor;
wherein at least one sensor of the module is positioned outside of the mouth and underneath the nares of a subject's nose when the upper and lower teeth trays are positioned in the subject's mouth.
